(19) United States
(12) Patent Application Publication
(10) Pub. No.: US 2023/0139440 A1
(43) Pub. Date: May 4, 2023

(54) Mouthpiece and method for intraoral treatment

(71) Applicant: Koite Health Oy, Espoo (FI)

(72) Inventors: Sakari Nikinmaa, Espoo (FI); Tommi Pätilä, Espoo (FI); Juha Rantala, Espoo (FI); Harri Joy, Espoo (FI)

(21) Appl. No.: 17/767,950

(22) PCT Filed: Oct. 12, 2020
(86) PCT No.: PCT/FI2020/050672

§ 371 Date: Apr. 11, 2022

(30) Foreign Application Priority Data
Oct. 11, 2019 (FI) 20195878

(51) Int. Cl. A61N 5/06; A61C 19/06

(52) U.S. Cl. CPC A61N 5/0603; A61N 5/062; A61C 19/066; A61N 5/0624; A61N 2005/007

(57) ABSTRACT

The invention relates to a mouthpiece and method for antibacterial treatment of intraoral surfaces. The mouthpiece comprises a body made of heat conducting material, the body comprising tilted facial and lingual outer surfaces and facial and lingual inner surfaces adapted to face facial and lingual surfaces of teeth, respectively. A light source is attached to the body's tilted surface and adapted to deliver light to surfaces of teeth, the light source being positioned on at least one of said outer surfaces and adapted to deliver said light via said inner surfaces to both the facial and lingual surfaces of the teeth.

### FIELD OF THE INVENTION

The invention relates to oral health care. In particular, the invention relates to a mouthpiece for antibacterial treatment of intraoral surfaces, such as teeth. The invention also relates to a method of delivering light within an intraoral photodynamic treatment mouthpiece and a method of treatment of intraoral tissues.

### BACKGROUND OF THE INVENTION

Oral health can be promoted by affecting bacteria inside the mouth. However, there is no effective way of managing oral bacterial load or the composition. More particularly there is no effective way to prevent or treat Streptococcus mutans, aka caries, bacterial infection. According to professional dentists, intraoral bacterial cleaning is required in every two months in order to keep the bacterial biofilm in check. Due to expertise and special equipment needed, this is currently impossible for most of the population.

In photodynamic treatment (PDT) oral bacteria are affected using light. PDT requires a lot of light energy, which generates e.g. heat build-up issues and the required intensity can also be hazardous for eyes. High energy levels are needed in particular in photosensitiser-augmented intraoral PDT, such as in indocyanine green (ICG) PDT.

One known intraoral treatment device is disclosed in U.S. Pat. No. 7,144,249 B2 and U.S. Pat. No. 6,616,447 B1. The device may comprise LED strip comprising LED lights covered by a transparent panel, which faces the facial surface of the teeth. There may also be provided a reflective panel behind the LED lights for maximizing the light energy targeted towards the teeth. The device is suitable for whitening the facial surface of the teeth. U.S. 8,371,853 B2 discloses another device suitable for the same purpose. Modifications of these device types and other existing intraoral treatment devices and methods are disclosed e.g. in U.S. 8,241,035 B2, U.S. Pat. No. 7,354,448 B2 and U.S. Pat. No. 9,572,645 B2.

U.S. Pat. No. 8,215,954 B2 discloses a mouthpiece comprising LEDs on the outside and inside the dental arch so as to allow for treatment of both facial and lingual surfaces of the teeth. The device may also comprise periscopic features that deliver light evenly vertically away from the LED to a teeth surface on the respective side.

Many of the existing solutions are based on delivering light only partially to teeth surfaces and/or with low light intensity, in which case the treatment efficiency in compromised. Otherwise there is a need of an extensive amount of LED components to cover all teeth surfaces, which for its part, increases the manufacturing cost of designs as well as cause heat build-up. Typically, low-price LEDs have lower thermal efficiency, whereby a balance between cost and heat build-up is difficult to find. Many of the proposed devices also suffer from poor suitability to mass production and poor safety. For example, in some designs there are LED components placed on the biting surfaces of the teeth, whereby there is a risk of breaking the LEDs by the user biting them in accident. Also the problem of danger of NIR light to the eyes has not been solved properly.

For example because of the abovementioned drawbacks, there are few viable consumer PDT products on the market. Efficient and safe PDT devices could, however, help make frequent antibacterial treatment available for ordinary consumers.

Thus, there is a need for improved intraoral treatment devices.

### SUMMARY OF THE INVENTION

It is an aim of the invention to solve at least some of the above-mentioned shortcomings and to provide a novel mouthpiece for intraoral antibacterial treatment.

It is a first object of the present invention to provide an oral treatment device comprising an intraoral mouthpiece.

It is a second of the present invention to provide a kit comprising an oral treatment device.

It is a third object of the present invention to provide a method of delivering light within an intraoral mouthpiece intended for photodynamic treatment of teeth and intraoral tissue.

It is a fourth object of the present invention to provide a method of treatment of intraoral tissues of a user.

The present invention is based on the idea of providing a mouthpiece comprising a body made of a heat conducting material. The body generally exhibits an upper surface and an opposite lower surface, which surfaces typically comprise an essentially planar portion between two opposite edges, at least one lingual edge and at least one buccal or labial edge. The mouthpiece is configured such that when the mouthpiece is in use, the surfaces will abut with teeth surfaces and optionally oral tissues. The upper and lower surfaces further contain a plurality of light sources attached to the body and adapted to deliver light to teeth surfaces.

The opposite edges of provided with surfaces, at least a part of which are planar and tilted so as to form in cross-section of the body an angle of up to 90° with respect to the planar portions of the upper and lower surfaces. At least a part of the light sources are preferably positioned on at least one of the tilted buccal, labial or lingual edges of the body. The body and the light sources are embedded in an encasing formed by a transparent polymeric material. Such material will protect the body while still allowing for light to pass through the encasing material to the teeth and tissues while the mouthpiece is placed in use position in the mouth of an individual.

In one embodiment, in the device the tilted edges form an angle of 5 to 75°, for example 10 to 65°, with regard to the planar portions of the upper and lower surfaces.

In one embodiment, the tilted edges form an angle of 10 to 55°, in particular 30 to 50°, with regard to the planar portions of the upper and lower surfaces.

A kit comprises an intraoral mouthpiece, preferably of the kind explained in the foregoing, with a body made of a heat conducting material combined with a control electronic circuit connected to the body, in particular connected to the body via a male USB port thereof.

A kit may also comprise an intraoral mouthpiece together with an intraoral agent, in particular an active agent selected from the group of benefit agents, including antioxidants, photosensitizers and potentiating agents and combinations thereof.

The present mouthpiece allows for the delivery of light for photodynamic treatment of teeth and intraoral tissue. In use, at least a part of the light sources are positioned on at least one of said tilted buccal, labial or lingual edges of the body, and the light sources are supplied with electric power for delivering light towards the user's teeth and intraoral tissue.

Thus, the mouthpiece in the user's mouth such that the upper and lower surfaces engage at least the chewing surface of the user's teeth; the light sources are supplied with electric power for generating light; and the light is directed towards the user's teeth at an angle with respect to the plane of the chewing surface of the teeth to achieve photodynamic oral treatment of the teeth and oral issue.

More specifically, the present invention is characterized by what is stated in the characterizing part of the independent claims.

Considerable advantages are obtained with the present technology. Embodiments of the present technology will contribute to improving efficiency of treatment and mitigate heat build-up, which typically and interrelated aims.

The present invention also provides for improved user safety and manufacturability.

The light sources are preferably LEDs (light emitting diodes) which will give high intensity of light at a preselected wavelength with low heat emission.

The present mouthpiece is capable of distributing light evenly to dental and periodontal locations, when a light source, such as a LED light source, is located at the facial or lingual surface of the teeth. By placing the light sources, in particular the LEDs facially or lingually, i.e. in the area between the lips or cheeks and the tooth, or between the teeth and the tongue, any thermal loss of the light source(s) will be absorbed by the surrounding tissues during the light exposure.

Furthermore, a placing of the light sources, such as LEDs, away from the occlusal surfaces of the teeth, reduces the risk of breaking the LED part by biting. Optical attributes of the modular elements in the mouthpiece enable an even light distribution on all surfaces of the teeth.

The LED elements may have an over 35% thermal efficacy.

Further features and advantages of various embodiments are disclosed in the following non-limiting description of embodiments.

### BRIEF DESCRIPTION OF THE DRAWINGS

FIG. 1 depicts in perspective view the mechanical structure of the encasing of the mouthpiece (uppermost drawing) and of the three overlapping trays inside the encasing according to an embodiment;

FIG. 2 shows a kit comprising a mouthpiece, a control unit, a local light head and a charging dock according to one embodiment;

FIG. 3 shows in perspective view a portion of an embodiment of a tray comprising rows of light emitting components placed along the rim of the tray in inclined position and some light emitting components placed in an embedded fashion on the biting surface of the tray; and

FIG. 4 shows a stand-alone implementation of a mouthpiece according to one embodiment.

### DESCRIPTION OF EMBODIMENTS

The present technology generally relates to mouthpieces and methods for antibacterial treatment of intraoral surfaces.

In various embodiments, the mouthpiece comprises a body made of heat conducting material, the body comprising tilted facial and lingual outer surfaces and facial and lingual inner surfaces adapted to face facial and lingual surfaces of teeth, respectively. A light source is attached to the body's tilted surface and adapted to deliver light to surfaces of teeth, the light source being positioned on at least one of said outer surfaces and adapted to deliver said light via said inner surfaces to both the facial and lingual surfaces of the teeth.

In one embodiment, an oral treatment device comprises an intraoral mouthpiece which comprises a body made of a heat conducting material which exhibits an upper surface and an opposite lower surface. The surfaces are configured such that there is, preferably on both opposite sides of the generally planar body, surfaces comprising a planar or essentially planar portion which is located between edges, on at least the outer side, preferably on both opposite sides.

Thus, in one embodiment, there is a lingual edge and a buccal and/or labial edge. The planar portion or preferably planar portions are configured to be placed against the surfaces of the user's teeth when the mouthpiece is placed in the mouth of the user between the teeth. Thus, the planar surfaces of the body will abut with the biting surfaces of the teeth.

The body further comprises at least one, but preferably a plurality of light sources attached to the body and adapted to deliver light to teeth surfaces. Generally, there are about 1 to 20 light sources on each lingual side and 1 to 10 light sources labially. By providing the body with a plurality of light sources, it is possible to ensure an even emission of light against the teeth and gum areas or other intraoral areas.

Furthermore, the edges, arranged on either or both sides of the planar portions comprise oblique surfaces or consist essentially of such surfaces. At least a part of or all of the edge surfaces are planar. The surfaces are further at least partially and tilted so as to form an angle of up to 90°, in particular about 5 to 85° with respect to the planar portions of at least one of the upper and lower surfaces. By placing at least a part of the light sources on at least one of the tilted buccal, labial or lingual edges of the body it is possible to emit light at an angle of up to 90°, in particular 5 to 85° against the teeth surfaces and the intraoral tissue, such as gum about the teeth.

It should be noted that the angle of the edges of the body can vary in different parts of the mouthpiece, being for example 5 to 30° in a first portion of the body and 25 to 60° in a second portion of the body.

The mouthpiece preferably comprises an encasing for the body and the light sources. In particular, the body and the light sources are embedded in an encasing formed by a transparent polymeric material.

In one embodiment, the shape of the body onto which the light sources are assembled allows provision of LEDs on the base surface of the body at an angle of 90° or less, but preferably 5° or more, to direct light towards the teeth. The adjustment of the treatment and light intensity can be done by directing the LEDs towards teeth or towards gum or other intraoral tissue by adjusting the angle in the base surface of the body

In one embodiment, the tilted edges form an angle of 10° or more, in particular more than 20°, ° or at least 25°, preferably at least 30 °, with regard to the planar portions of the upper and lower surfaces. Thus, in one embodiment, the tilted edges form an angle of more than 20° or at least 30° and up to 85°.

In one embodiment, the tilted edges form an angle of 10 to 55°, in particular 30 to 50°, with regard to the planar portions of the upper and lower surfaces.

The present technology also provides, in an embodiment, a method of delivering light within an intraoral mouthpiece intended for photodynamic treatment of teeth and intraoral tissue.

The method comprises the steps of providing a body made of a heat conducting material, the body further having an upper surface and an opposite lower surface, said surfaces comprising an essentially planar portion between lingual edges and buccal or labial edges, respectively, and configured to be placed against teeth surfaces.

At least a part of which are planar and tilted so as to form in cross-section of the body an angle of 5 to 85° with respect to the planar portions of the upper and lower surfaces.

In the method, there are positioned on at least a part of the edges of the body light sources to the body which are adapted to deliver light to teeth surfaces and intraoral tissue, and electric power is supplied to the light sources.

In a method of treatment of a user's teeth and intraoral tissue a mouthpiece as discussed above is placed in the user's mouth such that the upper and lower surfaces engage at least the chewing surface of the user's teeth; the light sources are supplied with electric power for generating light; and the light is directed towards the user's teeth at an angle, in particular an oblique angle, with respect to the plane of the chewing surface of the teeth to achieve photodynamic oral treatment of the teeth and intraoral tissue.

Further details of embodiments will appear from the drawings.

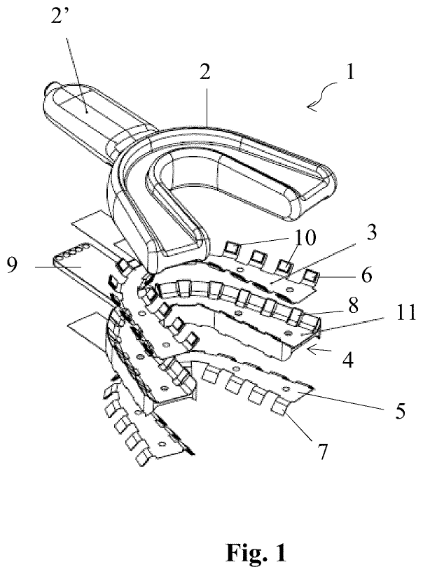

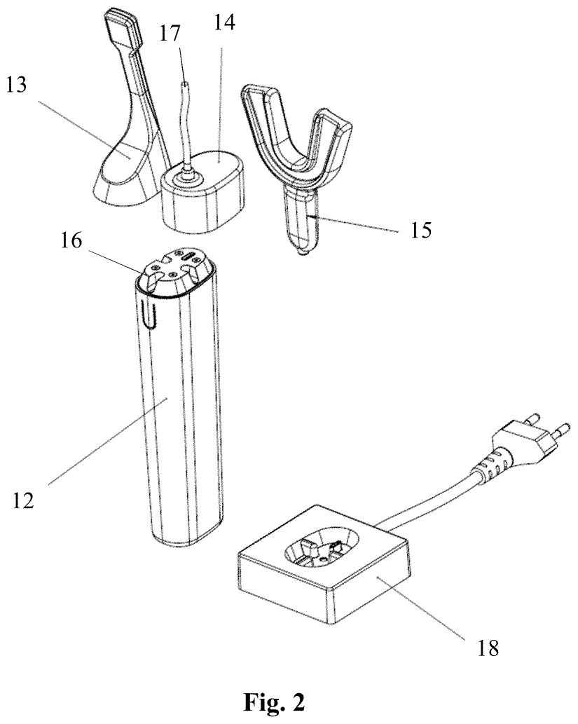

The following features typical of one embodiment are shown in FIG. 1:
A polymeric cover or encasing 2, which can comprise silicone rubber or thermoplastic, preferably transparent or translucent and approved for human use;
Sheet or layers 3, 5, preferably flexible and each comprising, in particular, a printed circuit board;
Light emitting components (light sources) 10, for example comprising light emitting diodes (abbreviated LEDs); and
A body in the form of a sheet or plate 4 which generally is U-shaped (in planar direction) and preferably rigid.

In the present context, the term “rigid” denotes that the body (i.e. sheet or plate) 4 is not deformed during normal use of the mouthpiece—for example it is not essentially bent in either longitudinal or transversal direction by the biting force exerted by the user of the mouthpiece.

The sheet or plate 4 comprises in particular a thermally conducting material.

Preferably, the body comprises a generally U-shaped arc (3-5) so as to be able to follow a dental arc.

Structure 4 will also be referred to in the following as a “tray” or a “heat dissipating” (or “hear conducting”) tray. The tray typically has an extended portion, in particular at the front end (in use position), which forms a cooling tray or surface 9.

Thus, in one embodiment, at the far end (i.e. bottom) of the U, on the opposite side to the legs of the U, extending generally in opposite direction to the legs, there is preferably an extended portion which in particular functions as a cooling surface or tray.

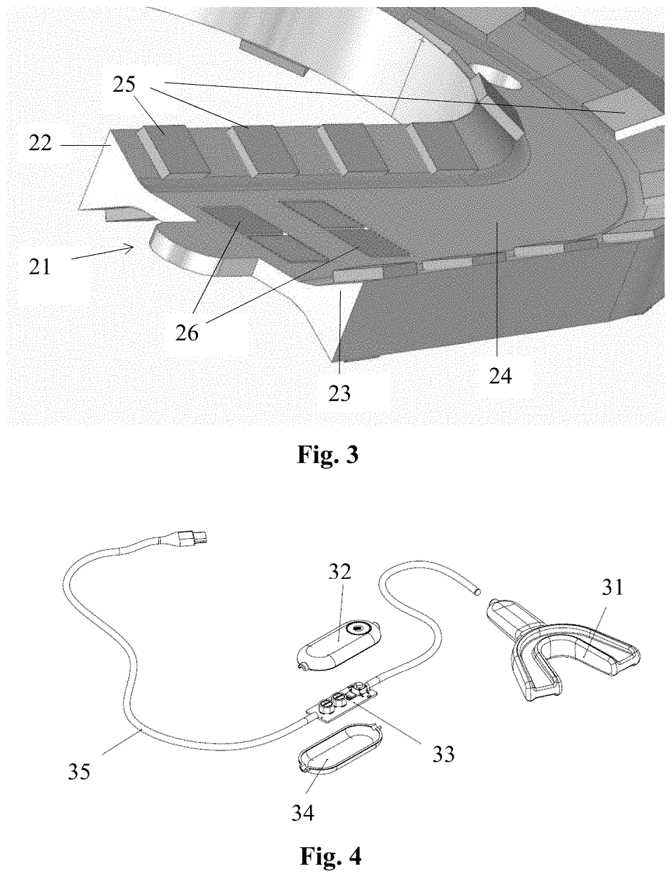

Similarly, FIG. 3 shows a part of a tray according to an embodiment, with reference numeral 21 generally referring to the tray and numeral 24 referring to the planar portion of the tray 21. Numerals 22 and 23 refer to the inclined edge portions of the tray and numerals 25 and 26 refer to light sources. As will appear, a first set of which (cf. numerals 25) are located on the inclined edge portions of the tray 21. A second set 26 is located on the planar portions 24 of the tray 21.

The tray typically comprises a body of metal or of a heat conducting polymer or a combination thereof. The preferred heat conduction (also known as “thermal conductivity”) for the material of the body (in the following also “base”) is greater the 0.5 W/mK. For metal bases it is typically between 1 and 1000 W/mK, preferably 5 to 400 W/mK, in particular 50 to 250 W/mK. The preferred heat conduction for a polymer base is between 0.5 to 100 W/mK, preferably 1 to 20 W/mK, such as 5 to 15 W/mK. The afore-going values stand for “thermal conductivity in plane” and can be determined by the ASTM E1461.

In one embodiment, the heat conducting material of the body has a thermal conductivity of greater than 0.5 W/mK, for example greater than 1 W/mK, in particular 1.1 to 25 W/mK for polymer materials and 100 to 500 W/mK for metal materials.

As can be seen in FIGS. 1 and 3, at least a part of the light sources 10; 22 are configured to deliver light at an angle, in particular an oblique angle, with respect to the plane 11; 24 of the tray 4; 21.

The tray 4; 21 will allow for the assembly of light sources into the mouth piece such that they are capable of directing light at an angle, in particular an oblique angle towards teeth. To that end, the tray 4; 21 comprises planar area portions 11; 24 defined between opposite edge portions 8; 22, 23 which are tilted or include with respect to the planar portions. As a result, as will appear from FIGS. 1 and 3, the tray at least partially exhibits a time glass shaped cross-section. As also will appear, the edges are tilted so as to form a slope towards the planar portions of the upper and lower surfaces.

In one embodiment, the body comprises light reflecting, light guiding or both light reflecting and light guiding elements positioned at the edges of the upper and lower surfaces of the body, said elements being capable of reflecting or guiding, or both, light emanating from said light sources.

In one embodiment the light emitting components can be embedded in the rigid tray to increase the light to biting surface and protect the component from mechanical stress. One possible option is to use two sided flexible circuit boards and have asymmetrical holes in the tray to allow light to travel to opposite side of the mouthpiece.

The adjustment of the treatment and light intensity can be done by adjusting the position of the light emitting surfaces of the light sources with respect to the teeth. Thus, the light sources can be configured to emit light towards the teeth or gum or both. It is also possible to adjust the positions of the supporting edges 6, 7; 22, 23 of the tray and/or to adjust the thickness of the polymer cover to adjust light distribution.

In one embodiment, the light sources provide light which is emitted against the teeth or gum or both at an acute or oblique angle. In one embodiment, the angle of the light emitting surfaces of the light sources varies between 0 and 90 degrees, preferably light is directed against the teeth or gum or both at an angle of about 5 and 85 degrees, for example between 30 and 60 degrees, and in particular 35 to 50 degrees. The angle refers to the direction of the incident light waves having the greatest energy of the light emitted by the light sources.

In one embodiment, when using LEDs or similar light sources which have an at least essentially light emitting surface, it is preferred that that surface be configured to emit light at an oblique angle, said angle preferably being about 5 to about 85°, with respect to a plane defined by the biting surfaces of the teeth. To that end, the light sources can be mounted on edge portions of the tray which stand at an oblique angle on either or both sides of a planar surface of the tray. In particular, such a planar surface is preferably configured to abut with the biting surfaces of the teeth when the mouthpiece is being used.

As shown in FIG. 3, the tray may further have light sources, such as LEDs 23, embedded into the biting surfaces to increase the light intensity directed to biting surface of the teeth.

The light sources can be configured as flexible printed circuit boards. Typically, the tray 4; 21 supports two strings of LEDs on each side of the tray as shown in FIG. 1.

The light sources may comprise a multi-LED strip positioned at and preferably arranged to follow the shape of the buccal, labial or lingual edges of the body.

The light sources can be are positioned on the buccal or labial side, or both, of the body. They can also be positioned on the lingual outer surface of the body.

In one embodiment, the light sources are adapted to deliver light to the tongue through the lingual outer surface of the body.

In one embodiment, the light sources, such as LEDs, used in the mouthpiece are capable of emitting light having an average power density of 30 to 1000 mW/cm², in particular 50 to 500 mW/cm², towards the teeth.

The LEDs can be connected to any kind of circuit board including, but not limited to, flexible PCB, copper strips, metal PCB, semi flexible PCB and combination of them.

The thermal junction between the tray and the light emitting element can be increased by thermal conductive material such as thermal paste or thermal conductive glue (not-shown in the drawings).

The tray 4; 21 dissipates heat from the hotspots around the mouth where the heat is dissipated in controlled way to oral surfaces of user's mouth. Allowing good light power and solving the issue of light emitting component overheating or formation of local hotspots.

As shown in the embodiment of FIG. 1, the part 9 of the tray 4 external to mouth can protrude out from the silicone cover and it can incorporate a passive or active cooling function 2′. The tray 4 can further have grooves or pipes to allow cooling liquid or air to travel inside the tray. Active cooling can comprise but is not limited to assisted air cooling, water cooling, oil cooling or Peltier cooling or combination of them. Passive cooling can comprise but not limited to air cooling via convection, heat pipe cooling, evaporative cooling or heat storage cooling, radiative cooling or combination of them.

Based on the above, in one embodiment in a device the heat conducting body 3-5, 9 contains an extended portion which forms a cooling tray or surface 9, which is capable of conducting heat from the body 3-5 inside an encasing 2 to the outside of the encasing.

Typically, the extended portion is placed at the front end of the tray (when the mouth piece is placed in position between the teeth).

In one embodiment, the tray is connected to an active cooling unit for cooling the body inside the encasing, said active cooling being connected to a temperature sensor to allow for adjustment of cooling of the body depending on the temperature of the mouthpiece.

The mouthpiece cover 2 can comprise or consist or consist essentially of a polymer material approved for oral use, such as silicone rubber or a thermoplastic material or a combination of them. The cover may comprise a single uniform layer or multiple layers of same of different material. The cover may also include optically active components or material which alter the optical properties of the mouthpiece or induce other effects such as antimicrobial effect through ROS production but not limited to it. Optically active compounds can belong but are not limited to TiO₂, SiO₂ doped derivatives of them such as derivatives doped with Au, Ag, N, Fe or Cu or combinations thereof.

In one embodiment, the polymeric material forming the encasing contains, mixed therewith or as a coating on the encasing, an antibacterial and/or light diffracting material, such as TiO₂, SiO₂, Ag, metal doped TiO₂, metal doped SiO₂, N-TiO₂, N-SiO₂ or silver nanoparticles or combinations thereof.

In the embodiment of the invention lacking embedded light emitting surfaces to biting surface, the thickness of mouthpiece cover at biting area of the teeth is over 1 mm, preferably 2-3 mm optimally up to 10 mm. A thickness of 5 to 10 mm is particularly interesting for soft materials.

In the embodiment of the invention with LEDs at biting surface 23 the thickness of the cover is over 0.1 mm preferably over 1 mm and up to 10 mm at the most.

Generally, the body embedded in the encasing is capable of enduring a temperature of up to 100° C. during the operation of the device.

To achieve the best treatment effect the invented mouthpiece should be used as a kit comprising an interchangeable mouthpiece and a local light illuminator head and a control unit and a charging dock to be used together with an active substance.

To achieve the best treatment effect the invented mouthpiece should be used as a kit comprising an interchangeable mouthpiece and a local light illuminator head and a control unit and a charging dock to be used together with an active substance.

Embodiments relate to mouthpieces comprising a body made of heat conducting material, the body comprising tilted facial and lingual outer surfaces and facial and lingual inner surfaces adapted to face facial and lingual surfaces of teeth, respectively. A light source is attached to the body's tilted surface and adapted to deliver light to surfaces of teeth, the light source being positioned on at least one of said outer surfaces and adapted to deliver said light via said inner surfaces to both the facial and lingual surfaces of the teeth.

In one embodiment, the light sources are configured to emit simultaneously both non-visible treatment light and visible safety light, the visible safety light preferably having intensity greater than 1.8 cd, preferably greater than 25 cd and even greater than 100 cd. The intensity is preferably equal to or smaller than 5000 cd, in particular equal to or smaller than 3000 cd, for example 1500 cd or less.

In one embodiment, the light sources are configured to emit light to achieve treatment intensity and treatment time based on sensory input, in particular based on the intraoral sensor.

Preferably at least a part of the light sources are capable of producing light having multiple peak wavelengths.

In one embodiment, at least a part of the light sources have multiple light emitting surfaces.

In one embodiment, the encasing includes bristles, shapes, rods capable of brushing teeth and electric motor capable of moving or vibrating bristles, shapes, and rods and combinations thereof.

Turning to FIG. 3 it can be noted that the light sources 10 are connectable to a power source 12, such as a built-in electric power reservoir, for powering the light sources.

As shown in the figure, a kit comprising an oral treatment device comprises a body made of a heat conducting material 15 and a control electronic circuit 14, 17 connected to the body, in particular the control electronic circuit is connected to the body via a male USB port thereof.

In one embodiment, the kit comprises an oral treatment device together with a control unit 14, 17, an LED head 13 and a docking station 11.

In one particular embodiment, the mouth piece of the oral treatment device is connectable to a USB-C port.

In one embodiment, the kit comprises intraoral agent, in particular an active agent selected from the group of benefit agents, including antioxidants, photosensitizers and potentiating agents and combinations thereof. Thus, for example, the intraoral agent can be selected from the group of antioxidant molecules, such as vitamin E or an analog or precursor thereof, pigments, and combinations thereof.

Specific examples of active agents, in particular intraoral agents comprise photosensitizers selected from the group of Hypericin, curcumin, phenalenone derivatives, Cercosporin, psoralen, xanthotoxin, Angelicin, alpha-Terthienyl, Phenylthepatriyne, THC, Cannabidiol (CBD). Synthetic photosensitizers include the following: RB (Rose Bengal), MB, Porphyrin derivatives, Curcumin derivatives, Methylene Blue, Indocynine Green, Erythosine, Phenalenone derivatives, Fullerene derivatives, Xanthene derivates, optionally together with a pigment.

In one embodiment, the intraoral agent is selected from indocyanine green in combination with titanium dioxide, optionally further combined with at least one antioxidant, such as E-vitamin or an analogue or precursor thereof.

In one embodiment, the mouthpiece comprising means for mechanically vibrating the body while positioned intraorally.

The mouthpiece may comprise a built-in electric power reservoir, such as a battery or supercapacitor, for powering the light source(s) and any other electronic functions that the mouthpiece incorporates. In some embodiments, the power reservoir can be charged wirelessly.

Power can be switched on for example automatically when disconnected from the charger or manually when the user bites the mouthpiece, as detected by a suitable sensor.

In one embodiment, the power storage is implemented using one or more supercapacitors which can drive the device at least for 1 minute, preferably at least for 2 minutes, for example 5 minutes and up to 120 minutes, in particular at least 15 minutes and up to 30 minutes. There may for example be one supercapacitor per one or a group of LEDs.

The device can also have one or more sensors. This allows for providing a measurement and/or feedback functionality to the treatment process. In some embodiments, at least one sensor is provided for measurement of light absorption of LED components from LED current. In alternative or supplementary embodiments, there may be provided at least one photosensitive element, such as a diode, capable of measuring light absorption. Light absorption information can be linked to amount of biofilm on top of teeth and to early onset of caries.

In some embodiments, the mouthpiece is provided with a sensor for measure change in absorbed light of the emitted wavelength or wherein the component associated with the sensor output emits light of a first wavelength, and the sensor input detects light of a second wavelength different than the first wavelength.

In some embodiments, the mouthpiece is provided with one or more temperature sensors adapted so measure the temperature of the light sources or their surroundings. There may also be means for continuous monitoring of the temperature and for limiting LED power if the temperature rises to a predefined level. This will keep the user safe and allow LEDs to stay in their optimal zone for best thermal efficacy.

In some embodiments, the mouthpiece surface is at least partially coated with small layer of titanium dioxide that will produce reactive oxygen as 405 nm light is emitted from the structure. Titanium dioxide can be used as the diffusive or diffracting material in the mouthpiece and it also has a therapeutic bacteria-killing function as such.

In some embodiments, the It is also possible to make light weight photodynamic treatment device for low effect local use by using only one or two modular elements with a limited number of electronic components. Charging of mouthpiece is done wirelessly to a super capacitor. The device lights up when lifted from charger.

In some embodiments, the mouthpiece is provided with a self-antibacterial function by having an antibacterial layer thereon and irradiating the antibacterial layer, for example periodically when in standby-mode, e.g. in a charger. For example, the mouthpiece may provide 405 nm light to titanium dioxide particles contained in the mouthpiece

In one exemplary use case, the treatment is started by applying the active ingredient product to mouth and then placing the mouthpiece into the mouth and connecting the mouthpiece with an external control unit by a cord or wirelessly. The control unit can be a mobile device which is easy to carry during the treatment. After completed treatment, the may take a probiotic product in form of gum, tablet, paste or liquid, for example.

In one embodiment, the mouthpiece comprises at least one sensor, such as an optical sensor, for measuring the response of intraoral tissue or intraoral agent to light emitted thereto, such as photobleaching of active ingredient or the amount of active ingredient in the treatment area.

In some embodiments, the present photodynamic therapy mouthpiece has one or more safety features that prevent user to harm him-/herself with high intensity invisible light. In one implementation, a safety feature is based on regulating the brightness of the light source(s) for light components outside of 390-700 nm wavelengths based on a sensor input of the device or include additional light source of emitting light in the visible 390-700 nm spectrum to generate the natural and active aversion response of the eye to bright light. The device may additionally be configured to turn from low power to high power state once detecting target wavelength from treatment area that is inherent to a target molecule. In some embodiments, the device utilizes 780-815 nm light as primary active light and bright white light as eye-protecting light.

In some embodiments, the device is configured such that it switches from low power to high power treatment radiation only after detecting fluorescence radiation from its target area by use of a sensor, and thus protection of the eyes and other tissues from unnecessary exposure to radiation is achieved.

In one embodiment, the light sources are configured to emit light within at least one non-visible wavelength band, such as within a band of 780-820 nm.

In one embodiment, the mouthpiece comprises an intraoral sensor and means for regulating the output power of the light sources on at least one wavelength, in particular at least one non-visible wavelength band, based on input from the intraoral sensor.

Further, in some embodiments, the means for regulating the output power of the light source are configured to increase power output of the light sources in response to detecting a particular wavelength by said intraoral sensor.

1. An oral treatment device comprising an intraoral mouthpiece (1) comprising
a body (3-5, 9) made of a heat conducting material, the body further having an upper surface (3) and an opposite lower surface (5), said surfaces comprising an essentially planar portion between lingual edges (7) and buccal or labial edges (6), respectively, and configured to be placed against teeth surfaces, and
a plurality of light sources (10) attached to the body and adapted to deliver light to teeth surfaces,
wherein
said edges comprise surfaces (6, 7), at least a part of which are planar and tilted so as to form in cross-section of the body an angle of 5 to 85° with respect to the planar portions of the upper (3) and lower surfaces (5),
at least a part of the light sources (10) are positioned on at least one of the tilted buccal, labial or lingual edges of the body (3-5, 9), and
the body and the light sources are embedded in an encasing (2) formed by a transparent polymeric material.

2. The device according to claim 1, wherein tilted edges (6, 7) form an angle of 10 to 55°, in particular more than 20° or at least 25°, in particular 30 to 50° with regard to the planar portions of the upper (3) and lower surfaces (5).

3. The device according to claim 1 or 2, wherein the edges (6, 7) are tilted so as to form a slope towards the planar portions of the upper and lower surfaces.

4. The device according to any of claims 1 to 3, wherein the polymeric material forming the encasing (2) contains, mixed therewith or as a coating on the encasing, an antibacterial and/or light diffracting material, such as TiO₂, SiO₂, Ag, metal doped TiO₂, metal doped SiO₂, N-TiO₂, N-SiO₂ or silver nanoparticles or combinations thereof.

5. The device according to any of the preceding claims, wherein the polymeric material comprises a transparent or translucent thermoplastic or thermosetting material, e.g. a silicon, polycarbonate or polyacrylate polymer.

6. The device according to any of the preceding claims, wherein the a body (3-5, 9) is made of a heat conducting material having a thermal conductivity of greater than 0.5 W/mK, for example greater than 1 W/mK, in particular 1.1 to 25 W/mK for polymer materials and 100 to 500 W/mK for metal materials.

7. The device according to any of the preceding claims, wherein the body (3-5) and the light sources (10) are at least partly encapsulated by an encasing (2) having the shape of a dental arch.

8. The device according to any of the preceding claims, wherein the body (10) is encapsulated by an encasing (2) with a thickness of the encasing in the biting area extending to 0.1 mm or greater, in particular 1 mm and up to 10 mm, for example 2 to 5 mm.

9. The device according to any of the preceding claims, wherein heat conducting body (3-5, 9) contains an extended portion which forms a cooling tray or surface (9) which is capable of conducting heat from the body (3-5) inside the encasing (2) to the outside of the encasing.

10. The device according to any of the preceding claims, wherein the body (3-5) embedded in the encasing (2) is capable of enduring a temperature of up to 100° C. during operation of the device.

11. The device according to claim 9 or 10, wherein the tray (9), for example through its extended portion, is connected to a passive cooling unit for cooling of the part of the body (3-5) fitted inside the encasing (2).

12. The device according to any of claims 9 to 11, wherein the tray (9), for example through its extended portion, is connected to an active cooling unit for cooling of the part body (3-5) fitted inside the encasing (2).

13. The device according to any of claims 9 to 12, wherein the tray is connected to an active cooling unit for cooling the body inside the encasing, said active cooling being connected to a temperature sensor to allow for adjustment of cooling of the body depending on the temperature of the mouthpiece.

14. The device according to any of the preceding claims, wherein the body comprises a generally U-shaped arc (3-5) so as to be able to follow a dental arc.

15. The device according to any of the preceding claims, wherein the body comprises light reflecting, light guiding or both light reflecting and light guiding elements positioned at the edges of the upper and lower surfaces of the body, said elements being capable of reflecting or guiding, or both, light emanating from said light sources.

16. The device according to any of the preceding claims, wherein the light sources (10) comprise a multi-LED strip positioned at and preferably arranged to follow the shape of the buccal, labial or lingual edges of the body (3-5, 9).

17. The device according to any of the preceding claims, wherein the light sources (10) are positioned on the buccal or labial side, or both, of the body.

18. The device according to any of the preceding claims, wherein the light sources (10) are positioned on the lingual outer surface of the body.

19. The device according to any of the preceding claims, wherein light sources (10) are also placed on at least a part of the planar portions of the upper and lower surfaces, such light sources being capable of facing the chewing surfaces of the teeth when the mouth piece is in use.

20. The device according to any of the preceding claims, wherein the body (3-5) has a time glass shaped cross-sectional profile, which is capable of covering the horizontal and the two vertical surfaces of the teeth at one or both lines of teeth, respectively.

21. The device according to any of the preceding claims, wherein the light sources (10) are capable to emit light at an average power density of 30 to 1000 mW/cm², in particular 50 to 500 mW/cm², towards the teeth.

22. The device according to any of the preceding claims, wherein the light sources (10) are adapted to deliver light to the tongue through the lingual outer surface of the body (3-5).

23. The device according to any of the preceding claims, comprising at least one sensor, such as an optical sensor, for measuring the response of intraoral tissue or intraoral agent to light emitted thereto, such as photobleaching of active ingredient or the amount of active ingredient in the treatment area.

24. The device according to any of the preceding claims, comprising means for mechanically vibrating the body while positioned intraorally.

25. The device according to any of the preceding claims, wherein the light sources (10) are configured to emit light within at least one non-visible wavelength band, such as within a band of 780-820 nm.

26. The device according to any of the preceding claims, comprising an intraoral sensor and means for regulating the output power of the light sources (10) on at least one wavelength, in particular at least one non-visible wavelength band, based on input from the intraoral sensor.

27. The device according to claim 24, wherein said means for regulating the output power of the light source are configured to increase power output of the light sources (10) in response to detecting a particular wavelength by said intraoral sensor.

28. The device according to any of the preceding claims, wherein the light sources (10) are configured to emit simultaneously both non-visible treatment light and visible safety light, the visible safety light preferably having intensity greater than 1.8 cd, preferably 25 cd, and even greater than 100 cd.

29. The device according to any of the preceding claims, wherein the light sources (10) are configured to emit light to achieve treatment intensity and treatment time based on sensory input, in particular based on the intraoral sensor.

30. The device according to any of the preceding claims, where at least a part of the light sources (10) are capable of producing light having multiple peak wavelengths.

31. The device according to any of the preceding claims, wherein at least a part of the light sources (10) have multiple light emitting surfaces.

32. The device according to any of the preceding claims, wherein the light sources (10) are connectable to a power source (12), such as a built-in electric power reservoir, for powering the light sources.

33. The device according to claims 32, wherein the power reservoir is implemented using one or more supercapacitors, which are capable of operating the device at least for 1 minute, preferably at least for 2 minutes, for example 5 minutes and up to 120 minutes, in particular at least 15 minutes and up to 30 minutes.

34. The device according to any of the preceding claims, wherein the encasing (2) including bristles, shapes, rods capable of brushing teeth and electric motor capable of moving or vibrating bristles, shapes or rods or combinations thereof.

35. A kit comprising an oral treatment device according to any of claims 1 to 34, comprising a body made of a heat conducting material (15) and a control electronic circuit (14, 17) connected to the body, in particular connected to the body via a male USB port thereof.

36. The kit according to claim 35, comprising an oral treatment device according to any of claims 1 to 19, together with a control unit (14, 17), an LED head (13), a docking station (11).

37. The kit according to claim 35 or 36, wherein the mouth piece of the oral treatment device is connectable to a USB-C port.

38. The kit according to any of claims 35 to 37, comprising an intraoral agent, in particular an active agent selected from the group of benefit agents, including antioxidants, photosensitizers and potentiating agents and combinations thereof.

39. The kit according to claim 38, wherein the intraoral agent is selected from the group of antioxidant molecules, such as vitamin E or an analog or precursor thereof, pigments, and combinations thereof.

40. The kit according to claim 38 or 39, wherein the intraoral agent comprises a photosensitizer selected from the group of Hypericin, curcumin, phenalenone derivatives, Cercosporin, psoralen, xanthotoxin, Angelicin, alpha-Terthienyl, Phenylthepatriyne, THC, Cannabidiol (CBD). Synthetic photosensitizers include the following: RB (Rose Bengal), MB, Porphyrin derivatives, Curcumin derivatives, Methylene Blue, Indocynine Green, Erythosine, Phenalenone derivatives, Fullerene derivatives, Xanthene derivates, optionally together with a pigment.

41. The kit according to claim 40, wherein the intraoral agent is selected from indocyanine green in combination with titanium dioxide, optionally further combined with at least one antioxidant, such as E-vitamin or an analogue or precursor thereof.

42. A method of delivering light within an intraoral mouthpiece intended for photodynamic treatment of teeth and intraoral tissue, comprising
providing a body made of a heat conducting material, the body further having an upper surface and an opposite lower surface, said surfaces comprising an essentially planar portion between lingual edges and buccal or labial edges, respectively, and configured to be placed against teeth surfaces,
attaching a plurality of light sources to the body, and
adapting the light sources to deliver light to teeth surfaces and intraoral tissue,
further comprising
providing on the edges, at least a part of which are planar and tilted so as to form in cross-section of the body an angle of 5 to 85° with respect to the planar portions of the upper and lower surfaces,
positioning at least a part of the light sources on at least one of said tilted buccal, labial or lingual edges of the body, and
supplying the light sources with electric power for delivering light towards the user's teeth and intraoral tissue.

43. A method of treatment of intraoral tissues of a user, comprising
providing a mouthpiece according to any of claims 1 to 34;
placing the mouthpiece in the user's mouth such that the upper and lower surfaces engage at least the chewing surface of the user's teeth;
supplying the light sources with electric power for generating light; and
directing the light towards the user's teeth at an angle with respect to the plane of the chewing surface of the teeth to achieve photodynamic oral treatment of the teeth and oral issue.
